(19) United States
(12) Patent Application Publication
(10) Pub. No.: US 2003/0036531 A1
(43) Pub. Date: Feb. 20, 2003

(54) Use of lysophospholipids to inhibit mammalian phospholipase D

(75) Inventors: Jiwan P. Palta, Madison, WI (US); Stephen B. Ryu, Buk-gu (KR)

Correspondence Address: WOOD, PHILLIPS, KATZ, CLARK & MORTIMER, 500 W. MADISON STREET, SUITE 3800, CHICAGO, IL, 60661, US

(73) Assignee: Wisconsin Alumni Research Foundation

(21) Appl. No.: 10/145,458

(22) Filed: May 14, 2002

Related U.S. Application Data
(60) Provisional application No. 60/291,597, filed on May 16, 2001.

(51) Int. Cl. A61K 31/685; A61K 31/66

(52) U.S. Cl. 514/78; 514/114

(57) ABSTRACT

The present invention relates to the use of certain lysophospholipids to inhibit phospholipase D activity and the growth of tumor cells in a mammal.

### RELATED APPLICATION INFORMATION

This application claims priority from U.S. Application No. 60/291,597 filed on May 16, 2001.

### GOVERNMENT SUPPORT INFORMATION

This invention was made with United States government support awarded by the following agencies:

USDA 93-37100-8924

The United States has certain rights in this invention.

### TECHNICAL FIELD OF THE INVENTION

The present invention relates to the use of certain lysophospholipids to inhibit phospholipase D activity and the growth of tumor cells in a mammal.

### BACKGROUND OF THE INVENTION

Phospholipase D (hereinafter “PLD”) has been implicated in signal transduction, regulation of inflammatory and immune responses, cellular trafficking, and cell growth (see Exton, J. H., J. Biol. Chem., 265:1-4 (1990), Exton, J. H., Biochim. Biophys. Acta, 1212: 26-42 (1994), Exton, J. H., Biochim. Biophys. Acta 1436:105-115 (1998), Shukla, S. D., et al., Life Sci. 48:851-866 (1991), Gomez-Cambronero, J., et al., Cellular Signalling, 10:387-397 (1998)). Specifically, PLD is believed to play an important role in tumor promotion and cell proliferation. PLD catalyzes the hydrolysis of phospholipids to yield phosphatidic acid (hereinafter “PA”) and the free polar headgroups. PA has been implicated as a biologically active molecule and has been found to be metabolized by a PA phosphohydrolase to form diacylglycerol, a protein kinase C activator (see Exton, J. H., Biochim. Biophys. Acta, 1212: 26-42 (1994), Gustavsson, L., et al., J. Biol. Chem, 269:849-859 (1994), Billah, M. M., et al., J. Biol. Chem., 264:17069-17077 (1989), Conricode, K. M., et al., J Biol. Chem., 267:7199-7202 (1992), Conricode, K. M., et al., FEBS Letters, 342: 149-153 (1994), Moehren, G., et al., J Biol. Chem. 269:838-848 (1994)). When added exogenously to cells, PA has been found to stimulate phospholipase A₂ activity (see Hashizume, T., et al., Biochim. Biophys. Acta, 1221:179-184 (1994), Bauldry, S. A., et al., Biochem. J, 322:353-363 (1997)).

Several studies have focused on how PLD activity is regulated. While PLD-activating mechanisms have been widely studied, the negative regulation of PLD is poorly understood. This is primarily due to the lack of known specific inhibitors of PLD (see Cockcroft, S., Chem. Phys. Lipids, 80:59-80 (1996)). The existence of PLD-inhibitory factors has been found in several mammalian tissues (see Brown, H. A., et al., J. Biol. Chem., 270: 14935-14943 (1995), Geny, B., et al., Eur. J. Biochem., 231:31-39 (1995), Kim, J. H., et al., J. Biol. Chem., 271: 25213-25219 (1996), Han, J. S., et al., J. Biol. Chem. 271:11163-11169 (1996),Venable, M. E., et al., J. Biol. Chem., 271:24800-24805 (1996), Kiss, Z., et al., FEBS Letters, 412:313-317 (1997)). Some of the protein factors in brain cytosol that exhibited a potent inhibitory action on PLD have been identified: fodrin (see Lukowski, S., et al., J. Biol. Chem., 271: 24164-24171 (1996)), synaptojanin (see Chung, J. K., et al., J. Biol. Chem., 272:15980-15985 (1997)). and clathrin assembly protein 3 (see Lee C., et al., J. Biol. Chem., 272:15986-15992 (1997)). A ceramide was also reported to inhibit PLD activation by phorbol myristate acetate by interfering with protein kinase C (see Venable, M. E., et al., J. Biol. Chem., 271:24800-24805 (1996)). ADP-ribosylation factor (hereinafter “Arf”)-related protein was recently found to inhibit Arf-dependent activation of PLD by binding to the Arf-specific guanine nucleotide exchange factor cytohesin (Schürmann, A., et al., J. Biol. Chem., 274:9744-9751 (1999)). It was recently found that lysophospholipids such as lysophosphatidylethanolamine (hereinafter “LPE”) and lysophosphatidylinositol (hereinafter “LPI”) inhibit plant PLD in a highly specific manner (see Ryu, S. B., et al., Proc. Natl. Acad. Sci. USA, 94:12717-12721 (1997)). Furthermore, the lysophospholipids were found to function as lipid-derived growth regulators involved in retarding senescence of plant tissues (see Farag, K. M., et al., Physiol. Plant, 87:515-524 (1993), Ryu, S. B., et al., Proc. Natl. Acad. Sci. USA, 94:12717-12721 (1997)) and in stimulating plant cell growth (see Scherer, G. F. E., Plant Growth Regulation, 18:125-133 (1996), Scherer, G. F. E., et al., Planta, 202:462-469 (1997)). Another recent study observed the presence of PLD inhibitors in pig colon microsome extract, and these were identified to be lysophosphatidylserine (LPS), LPI, and phosphatidylinositol (Kawabe, K., et al., J. Biochem., 123: 870-875 (1998)).

### SUMMARY OF THE INVENTION

In one embodiment, the present invention relates to a method for inhibiting phospholipase D activity in a mammal. The method involves administering to a mammal an effective amount of a composition to inhibit phospholipase D activity. The composition contains at least one lysophospholipid selected from the group consisting of lysophosphatidylglycerol, lysophosphatidylinositol and lysophosphatidylserine. The composition used in this method can be administered to a mammal intravenously, subcutaneously, orally or topically.

In a second embodiment, the present invention relates to a method for inhibiting the growth of tumor cells in a mammal. The method involves administering to a mammal an effective amount of a composition to inhibit the growth of tumor cells. The composition contains at least one lysophospholipid selected from the group consisting of lysophosphatidylglycerol, lysophosphatidylinositol and lysophosphatidylserine. The composition used in this method can be administered to a mammal intravenously, subcutaneously, orally or topically. Additionally, the composition can be used to inhibit the growth of prostate carcinoma tumor cells, melanoma tumor cells, glial brain tumor cells, Kaposi's sarcoma tumor or lymphoma tumor cells, lung adenocarcinoma tumor cells, breast cancer tumor cells, osteosarcoma tumor cells, fibrosarcoma tumor cells, or squamous cancer tumor cells.

### BRIEF DESCRIPTION OF THE DRAWING

FIG. 1 shows the effect of lysophospholipids on rat brain PLD activity. The activity of partially purified PLD from rat brain membrane was assayed using the standard reaction procedures described in Example 1 in the presence of different compounds of which the final concentration was 10 μM. The standard reaction mixture contained PLD stimulators such as 4.8 μM phosphoinositol 4,5-bisphosphate (hereinafter “PIP₂”), 100 nM Arf, and 5 μM guanosine 5′-O-(3-thio)-triphosphate (hereinafter “GTPγS”) (“bLPE” refers to brain LPE and “eLPE” refers to egg LPE). Data points were mean±SEM of three separate experiments.

FIG. 2 shows the inhibition of rat brain PLD activity as a function of lysophospholipid concentration. The activity of partially purified brain PLD was assayed using the standard reaction procedures described in Example 1 in the presence of different concentrations of, lysophosphatidylinositol (hereinafter “LPI”) (●) and lysophosphatidylserine (hereinafter “LPS”) (ρ). Half-maximal inhibition of PLD activity was observed at 3 μM LPI and 6 μM LPS. Data points were mean±SEM of three separate experiments.

FIG. 3 is a Lineweaver-Burk plot suggesting the noncompetitive inhibition of rat brain PLD by lysophospholipids. The Lineweaver-Burk plot was obtained by measuring the activity of partially purified rat brain PLD at different concentrations of a substrate lipid mixture of phosphatidylethanolamine (hereinafter “PE”), PIP₂ and phosphatidylcholine (hereinafter “PC”) in a molar ratio of 16:1.4:1 in the absence (o) and presence of inhibitors such as 4 μM LPI (●) and 4 μM LPS (π). V indicates reaction velocity (pmol/h) and [S] a substrate concentration (μM) of PC. Results are shown as data points representing three separate experiments.

FIG. 4 shows that the inhibition of rat brain PLD by lysophospholipids is not due to their interaction with Arf. In FIG. 4A, the control, the activity of partially purified rat brain PLD was assayed by standard reaction procedures as described in Example 1, while ‘−Arf’ indicates that the PLD activity was assayed in the absence of Arf from the standard reaction mixture. ‘−Arf +LPI’ represents that the PLD activity was assayed in the absence of Arf but in the presence of the inhibitor, LPI (4 μM). In FIG. 413, the control (o), the activity of partially purified rat brain PLD was assayed using the standard reaction procedures described in Example 1 using the different concentrations of myristoylated Arf. ‘LPI’ (●) indicates that the PLD activity was assayed in the presence of the inhibitor, LPI (15 μM). Data points were mean±SEM of three separate experiments.

FIG. 5 shows that the inhibition of rat brain PLD by lysophospholipids is not affected as the concentration of PIP₂ increases. In the control, the activity of partially purified rat brain PLD was assayed using standard reaction procedures described in Example 1 in the presence of two different concentrations of PIP₂: 1×PIP₂ (4.8 μM) and 3×PIP₂ (14.4 μM). ‘LPI’ indicates that the PLD activity was assayed in the presence of the inhibitor, LPI (4 μM). Data points were mean±range of two separate experiments.

FIG. 6 shows the binding of rat brain PLD to lysophospholipid vesicles. Rat brain PLD protein was incubated with lysophospholipid or phospholipid vesicles such as brain LPE (Lane 1), brain PE (Lane 2), and LPI (Lane 3) in the presence of an 1,500 fold excess of BSA as described in Example 1. After centrifugation, the PLD protein co-precipitated with lipid vesicles was subjected to electrophoresis on an 8% SDS-PAGE. PLD was visualized by alkaline phosphatase-immunoblotting using rabbit PLD 1 and −2 antibodies.

FIG. 7 shows the specificity of lysophosphatidylinositol in inhibiting the growth of cells from a prostate cancer cell line (hereinafter “PC-3”) compared to phosphatidylinositol and inositol.

FIG. 8 shows the effects of lysophospholipids on the proliferation of PC-3 cells. The results demonstrate that lysophosphatidylglycerol (hereinafter “LPG”), lysophosphatidylinositol and lysophosphatidylcholine (hereinafter “LPC”) have unexpectedly good results when compared to other lysophospholipids.

FIG. 9 shows the dose-dependency of lysophosphatidylinositol inhibition on PC-3 cell growth.

FIG. 10 shows the effects of perillyl alcohol (hereinafter “PerOH), a natural product currently used in treating cancer, on the proliferation of PC-3 cells.

### DETAILED DESCRIPTION OF THE INVENTION

In one embodiment, the present invention relates to a method of inhibiting phospholipase D activity in a mammal. The method involves administering to a mammal, such as, but not limited to, a human, an effective amount of a composition containing at least one lysophospholipid selected from the group consisting of lysophosphatidylglycerol, lysophosphatidylinositol and lysophosphatidylserine to inhibit phospholipase D activity. In a second embodiment, the present invention relates to a method of inhibiting the growth of tumor cells. The method involves administering to a mammal, such as, but not limited to, a human, a composition containing at least one lysophospholipid selected from the group consisting of lysophosphatidylglycerol, lysophosphatidylinositol and lysophosphatidylserine to inhibit the growth of said tumor cells.

U.S. Pat. No. 4,372,949 describes a method of treating a host mammal having cancer by administering to a mammal a therapeutically effective amount of a composition containing a lysophospholipid, such as lysophosphatidylcholine, and a phospholipid. However, the inventors of the present invention have discovered that a composition containing at least one of the following lysophospholipids, namely, lysophosphatidylglycerol, lysophosphatidylinositol or lysophosphatidylserine, is more efficacious in inhibiting phospholipase D activity and the growth of tumors cells then compositions containing other lysophospholipids, including lysophosphatidylcholine. These findings were surprising in view of the fact that the levels of lysophosphatidylglycerol, lysophosphatidylinositol and lysophosphatidylserine found in mammals is extremely low, if not entirely absent. For example, lysophosphatidylglycerol is a plant-specific lysophospholipid found only in the chloroplasts of plants. Thereupon, the discovery that a plant specific lysophospholipid is highly efficacious in inhibiting phospholipase D activity and the growth of tumor cells in a mammal was unexpected.

In a second embodiment, the composition described herein can be used to treat the growth of tumor cells in a mammal. The method involves administering to a mammal an effective amount of hereinbefore described composition to inhibit the growth of the tumor cells. The tumor cells that can be treated using this method include, but are not limited to, prostate carcinoma tumor cells, melanoma tumor cells, glial brain tumor cells, Kaposi's sarcoma tumor or lymphoma tumor cells, lung adenocarcinoma tumor cells, breast cancer tumor cells, osteosarcoma tumor cells, fibrosarcoma tumor cells, or squamous cancer tumor cells.

The lysophosphatidylglycerol, lysophosphatidylinositol and/or lysophosphatidylserine contained in the composition used in the methods described herein can be isolated and purified from a natural products or sources, such as a plant chloroplast, can be purified using routine techniques known in the art (see Bligh, E. G., et al., Canadian J. Biochem. and Physiol., 37(8):911-917 (August 1959), Lynch, D. V., et al., Plant Physiol., 74:198-203 (1984), Vigh, L., et al., Plant Physiol., 79:756-759 (1985)) or can be purchased from a lysophospholipid supplier such as Avanti Polar Lipids (Alabaster, Ala.). The amount of lysophosphatidylglycerol, lysophosphatidylinositol and/or iysophosphatidylserine contained in the composition is from about 20 to about 100 μM.

The lysophosphatidylglycerol, lysophosphatidylinositol and/or lysophosphatidylserine contained in the compositions exist in the D-, L- or DL-forms. Any of these forms can be used in the compositions described herein. Preferably, however, the L-form is used.

In addition to the lysophosphatidylglycerol, lysophosphatidylinositol and/or lysophosphatidylserine, the composition can also contain other additives which are commonly used in medicinal compositions, such as isotonic agents, such as glycerin, sorbitol, xylitol, sodium chloride, dextrose, etc., antioxidants such as vitamin A, vitamin B or the like, cholesterol, stearylamine, dicetylphosphate, dextran, methionine, glutathione or the like according to purpose and use.

The lysophosphatidylglycerol, lysophosphatidylinositol and/or lysophosphatidylserine contained in the compositions used in the methods of the present invention can be dispersed in the form of micells or lipid vesicles. Preferably, these dispersed particles have a particle size no larger than about 1.0 mμ.

At the time of use, the composition described herein can be administered in any one of a number of different forms. For example, the composition can be administered orally, such as in the form of a tablet, capsule, powder, granule or liquid, or as a sterilized liquid parenteral agent such as a solution or suspension. Solid preparations can be prepared as they are, as forms of tablets, capsules, granules or powders, or can be prepared using suitable additives. Suitable additives are well known in the art, and include, saccharides such as lactose and glucose, starches such as corn, wheat and rice, fatty acids such as stearic acid, inorganic salts such as aluminium magnesium metasilicate and anhydrous calcium phosphate, synthesized polymers such as polyvinylpyrrolidone and polyalkylene glycol, fatty acid salts such as calcium stearate and magnesium stearate, alcohols such as stearyl alcohol and benzyl alcohol, synthesized cellulose derivatives such as, methylcellulose, carboxymethylcellulose, ethylcellulose and hydroxypropylmethylcellulose, and further, water, gelatin, talc, vegetable oils, gum arabic, etc. Liquid preparations can be prepared as suspensions, syrups or injections using suitable additives typically used in liquid preparations such as water, alcohols or oils originated in vegetables such as, soybean oil, peanut oil and sesame oil. Particularly, solvents suitable in case of parenteral administration by intramuscular injection, intravenous injection or subcutaneous injection include, for example, distilled water for injection, aqueous lidocaine hydrochloride solutions (for intramuscular injection), physiological saline, aqueous glucose solutions, ethanol, liquids for intravenous injection (e.g. aqueous solutions of citric acid and sodium citrate, etc.), electrolyte solutions (for intravenous drip and intravenous injection), etc., and their mixed solvents. These injections can take such forms that powder itself or to which suitable additives were added is dissolved at the time of use, besides such forms that ingredients are dissolved in advance. The composition described herein can also be administered topically.

By way of example, and not limitation, examples of the present invention shall now be given.

### EXAMPLE 1

a. Materials

Bovine brain PC and PE were obtained from Avanti Polar Lipids, Inc. (Alabaster, Ala.), GTPγS and PIP₂ from Sigma, and 1,2-di[1-¹⁴C]palmitoyl PC from Amersham (Buckinghamshire, UK). Partially-purified PLD (classified as PLD I) from rat brain membranes and Arf purified from rat brain cytosol were obtained from Dr. Sue Goo Rhee (National Institutes of Health, Bethesda, Md.), and myristoylated Arf and rabbit PLD-antibodies were provided by Dr. Sung Ho Ryu (Pohang University of Science and Technology, Korea). Unless stated otherwise, the standard assay included Arf purified from rat brain cytosol. Lysophospholipids such as egg LPC, egg LPE, brain LPE, LPG (18:0), liver LPI, and brain LPS were obtained from Avanti Polar Lipids, Inc (Alabaster, Ala.). All other reagents were purchased from Sigma (St. Louis, Mo.).

b. Measurement of PLD Activity

PLD activity was measured according to optimum conditions as described in Han, J. -S., et al. J. Biol. Chem., 271:11163-11169 (1996) with a minor modification. A substrate lipid mixture of bovine brain PE, PIP₂, and bovine brain PC in a molar ratio of 16:1.4:1 with 1,2-di[1-¹⁴C]palmitoyl PC to yield 40,000 dpm per assay was emulsified in chilled water by sonication. To a standard enzyme assay mixture containing 50 mM Hepes (pH 7.5), 3 mM EGTA, 80 mM KCl, 2.5 mM MgCl₂, 2 mM CaCl₂, and 1% ethanol were added 100 nM Arf (rat brain), 5 μM GTPγS, 3 μl of PLD (58 ng), and 25 μl substrate (62.6 μM phospholipids) in a total volume of 150 μl. Assays were incubated at 37° C. for 30 minutes in a circulating water bath. The reaction was stopped by adding 750 μl chloroform:methanol (1:2). Chloroform (200 μl) was then added to the mixtures followed by 200 μl of KCl (2M). After vortexing, the chloroform and aqueous phases were separated by centrifugation at 12,000×g for 5 minutes. The chloroform phase was collected and dried under nitrogen gas. The samples were re-dissolved in 45 μl of chloroform and lipid standards were added before being spotted onto a thin layer chromatography plate (silica gel G). The plate was developed with a solvent containing chloroform:methanol:NH₄OH (65:35:5). Lipids on thin layer chromatography plates were visualized by exposure to iodine vapor and the spots corresponding to lipid standards were scraped into scintillation vials. Radioactivity in the scraped spots was quantitated by scintillation spectroscopy. The radioactivities of both products of transphosphatidylation activity (phosphatidylethanol) and hydrolysis activity (hereinafter “PA”) were combined to represent the activity of PLD (see Ryu, S. B., et al., Proc. Natl. Acad. Sci. USA, 94:12717-12721 (1997)).

c. Treatment of PLD Inhibitors

Lysophospholipids and free fatty acid (18:1) were dissolved in chloroform: methanol (95:5, v/v). After water was added, the organic solvents were removed by flowing nitrogen gas. Stock solution concentrations were adjusted to 250 μM with water before being added to the reaction mixture (Ryu, S. B., et al., Proc. Natl. Acad. Sci. USA, 94:12717-12721 (1997)). The range of the final concentrations of lysophospholipids ran from 40 nM to 20 μM. The control was assayed by adding solution, which had been prepared by expelling organic solvent in water. Headgroups such as choline, glycerol, inositol, and serine as well as triton X-100 were directly dissolved in water before being added to the reaction mixture. The results of PLD activity inhibition were expressed as a percentage of the control.

d. Measurement of Binding of Lysophospholipids to PLD

The binding ability of lysophospholipids to PLD was estimated by precipitation assay followed by immunoblot. The final assay volume was 0.3 ml containing 50 mM Hepes (pH 7.5), 3 mM EGTA, 80 mM KCl, 2.5 MM MgCl₂, 2 mM CaCl_{2, 1}% ethanol, 5 μM GTPγS, 33 nM myristoylated Arf, 300 μg of BSA, 200 ng of rat brain PLD and 1 mM lysophospholipid or phospholipid vesicles. After incubation at 37° C. for 10 minutes, the mixture was cooled in ice-water for 30 minutes and centrifuged at 17,000×g for 30 minutes at 4° C. The precipitant of lysophospholipid vesicles containing bound PLD in the excess of BSA (1,500 fold than rat PLD) was resuspended in 20 μL PBS (pH 7.5). The bound PLD was separated by 8% SDS-PAGE without reducing agent (2-mercaptoethanol) in sample loading buffer and transferred onto immunoblot membrane. The membrane was blotted with rabbit-PLD antibodies that detect both PLD 1 and PLD 2 isozymes. The PLD-antibody complex was visualized by staining alkaline phosphatase conjugated to a second antibody (Bio-Rad, Hercules, Calif.).

Results

i. Inhibition of Rat Brain PLD Activity by Lysophospholipids

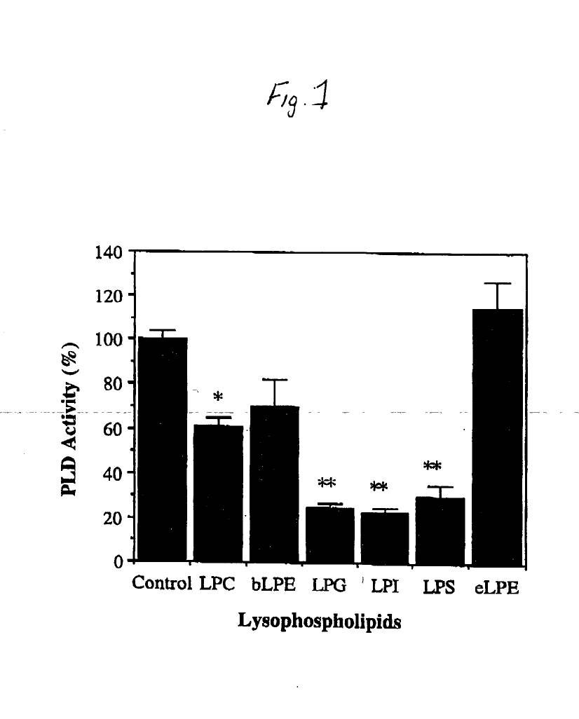

Different lysophospholipids were tested for their effects on the activity of partially purified rat-brain PLD. Among the lysophospholipids tested, LPI, LPG, and LPS had a potent inhibitory effect on PLD activity at 10 μM concentration, while LPC and brain LPE (hereinafter “bLPE”) showed some inhibitory effect (see FIG. 1). PLD activity was 23.3%, 24.9%, 30.3%, 61.0%, and 70.5% of the control in the presence of LPI, LPG, LPS, LPC, and bLPE, respectively. The PLD activity of the control was 25.3 nmol min⁻¹ mg⁻¹ protein. The ratio of two hydrolysis products (PA vs. phosphatidylethanol) was 0.83. The rat brain PLD activity was comparable to those reported previously in crude extract (0.3 pmol min⁻¹ mg⁻¹ protein) by Provost et al. (Provost, J. J., et al., Biochem. J., 319:285-291 (1996)) and in purified recombinant PLD 1 (100 to 200 nmol min⁻¹ mg⁻¹ protein) by Hammond et al. (Hammond, S. M., et al., J. Biol. Chem., 272: 3860-3868 (1997)). Brain LPE is composed of LPE (50%) and plasmalogen LPE (50%). When eLPE was tested for the inhibition of PLD, rat brain PLD activity was not inhibited but rather slightly stimulated (see FIG. 1). This result indicates that rat brain PLD may be inhibited by plasmalogen LPE, but not by LPE. Rat brain PLD was not stimulated by lysophosphatidic acid at the concentrations of 10 and 20 μM. LPA was previously found to stimulate PLD activity in PC-3 cells in vivo (Qi, C., et al., J. Cell. Physiol., 174:261-272 (1998)). However, the assay mixture contained other PLD stimulators—Arf and PIP₂. This result may suggest that PLD activity in vitro was not stimulated by LPA over and above the stimulation by Arf and PIP2.

Counter to the results with rat brain PLD presented here, the activity of PLD from plant tissues was not significantly inhibited by LPC, LPG or LPS, but only by LPE and LPI (Ryu, S. B., et al., Proc. Natl. Acad. Sci. USA, 94:12717-12721 (1997)). This difference suggests that animal and plant PLDs are regulated differently.

ii. Specificity of PLD Inhibition by Lysophospholipids

To resolve whether lysophospholipids inhibit rat brain PLD activity because of their detergent-like characteristics, the detergent triton X-100 was tested for its effect on PLD activity. Triton X-100 did not inhibit PLD activity, but did have a slight stimulatory effect (6-9%) when present at 10 μM concentration. Different headgroups and an acyl chain, subcomponents of lysophospholipids were tested. Free fatty acid (oleate) showed a slightly inhibitory effect (10%) at 20 μM concentration while significant inhibition of PLD by oleate (20%) was observed at 40 μM concentration. Hammond et al. (Hammond, S. M., et al., J. Biol. Chem., 270:29640-29643 (1995)) found that PLD1 was dramatically inhibited by oleate already at 10 μM concentration in absence of Arf and PIP2. Thus the lack of dramatic inhibition by oleate in these experiments is due to different assay conditions. None of the headgroups such as choline, glycerol, inositol, or serine significantly inhibited PLD activity at 10 μM concentration. These results demonstrate that rat brain membrane PLD is inhibited by lysophospholipids in a highly specific manner.

iii. Dose Dependency and Kinetics of the PLD Inhibition by Lysophospholipids

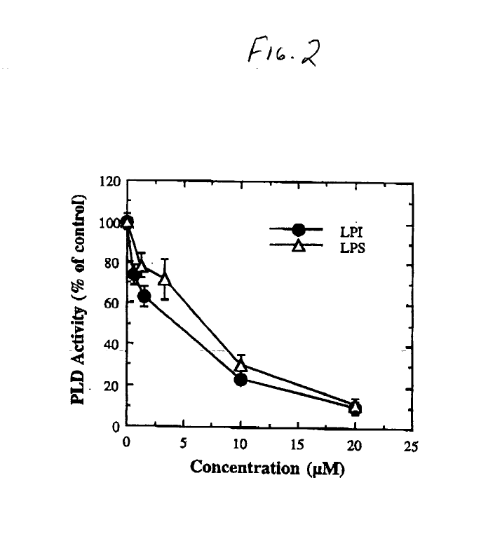

The activity of rat brain PLD was inhibited by lysophospholipids in a concentration-dependent manner. For example, the inhibitory effect of the lysophospholipids, LPI and LPS, on PLD activity was dose-dependent in the range of concentrations from 160 nM to 20 μM (see FIG. 2). LPI was a slightly more effective inhibitor than LPS as it was able to inhibit 19% of PLD activity at 160 nM and 27% at 640 nM. Half-maximal inhibition of PLD activity was observed at 3 μM LPI and 6 μM LPS.

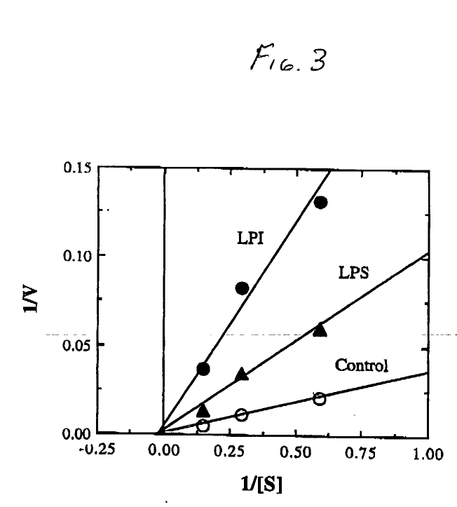

A Lineweaver-Burk plot was constructed to determine the effect of substrate concentration on the inhibition of PLD by lysophospholipids. The Kₘ for PLD was 33.3 μM PC and Vₘₐₓ was 166.7 pmol/h (see FIG. 3). The K^{m} did not change in the presence of the inhibitor LPI (4 μM) or LPS (4 μM); however, the Vₘₐₓ decreased to 121.5 pmol/h in the presence of LPS, and to 76.9 pmol/h in the presence of LPI (see FIG. 3). These results demonstrate that these lysophospholipids are non-competitive inhibitors of PLD.

iv. Inhibition of PLD by Lysophospholipids is Independent of PIP₂ and Arf

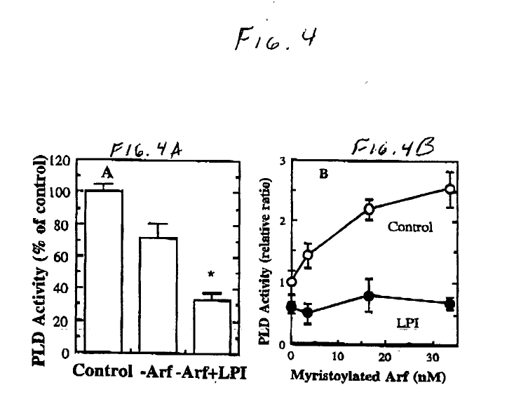

To address whether or not Arf is involved in the inhibition of rat brain PLD activity by lysophospholipids, the effect of lysophospholipids on PLD activity in the absence of Arf was examined. In the absence of Arf, PLD activity decreased to 72% of the control with Arf (see FIG. 4A). Adding the inhibitor LPI (4 μM) in the absence of Arf to the reaction mixture further reduced the PLD activity to 33% of the control (see FIG. 4A). Since rat brain PLD activity can be inhibited by LPI in the presence or absence of Arf, the inhibition may not be caused by an interference of LPI with Arf stimulation. In an earlier study, a protein factor from bovine brain cytosol was found to suppress PLD activity and this suppression was largely eliminated by the addition of Arf, suggesting that this protein inhibitor interacts with Arf (see Kim, J. H., et al., J. Biol. Chem., 271:25213-25219 (1996)). When rat brain Arf (100 nM) in a standard PLD assay of this study was substituted with 33 nM myristoylated Arf (Kim, J. H., et al., J. Biol. Chem., 271: 25213-25219 (1996)), PLD activity dramatically increased to 250% of the control (see FIG. 4B). However, in the presence of LPI (15 μM), PLD activity was not significantly changed by the addition of myristoylated Arf, indicating that Arf could not nullify the inhibition of PLD by LPI.

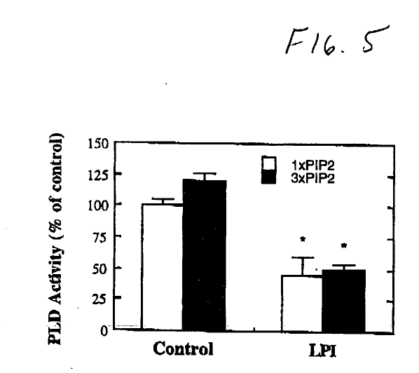

Since PLD activity requires PIP₂ as a cofactor, another possible means by which lysophospholipids can inhibit PLD is by interacting with PIP₂. For example, Clostridium difficile toxin B (Schmidt, M., et al., European J. Biochem., 240: 707-712 (1996)) and synaptojanin (Chung, J. K., et al., J. Biol. Chem., 272:15980-15985 (1997)) were found to inhibit PLD activity by depleting PIP². Simply by increasing theconcentration of PIP² PLD activity could be restored (Schmidt, M., et al., European J. Biochem. 240:707-712 (1996)). When the concentration of PIP₂ in the reaction mixture was tripled, the PLD inhibition by LPI (4 μM) was not significantly changed (see FIG. 5). When this experiment was repeated using LPS (12 μM), PLD inhibition by LPS was similar regardless of PIP₂ concentrations. These results demonstrate that inhibition of PLD by lysophospholipids is independent of PIP₂.

v. Binding of Lysophospholipids to Rat Brain PLD

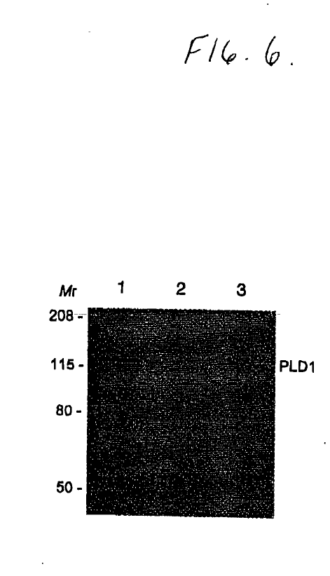

In view of the above results, the inhibition of PLD appears to be caused by the direct interaction of lysophospholipids with PLD rather than their disruption of PIP₂-PLD or Arf-PLD interactions. The binding capability of lysophospholipids to PLD itself was therefore determined. In the presence of an 1,500 fold excess BSA, PLD proteins co-precipitated with brain LPE or LPI vesicles, but not with brain PE vesicles (see FIG. 6). The distribution of precipitated protein bands on SDS-PAGE followed by Western blot with rabbit PLD1 and −2 antibodies (see FIG. 6) was similar to one of the original non-precipitated PLD preparation. A major band was detected at 120 kDa in the Western blot, indicating that the rat brain PLD preparation consists of PLD 1 but not PLD 2. The PLD preparation also contained some lower molecular weight proteins that could be detected by the PLD antibodies, suggesting partial proteolysis of some of the PLD protein. The co-precipitation of PLD proteins by iysophospholipid vesicles suggests direct binding between PLD and lysophospholipids. The fact that this precipitation of PLD did not occur with the phospholipid vesicles further supports this argument.

### EXAMPLE 2

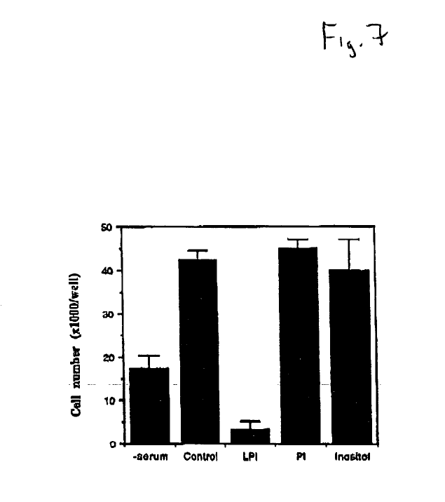

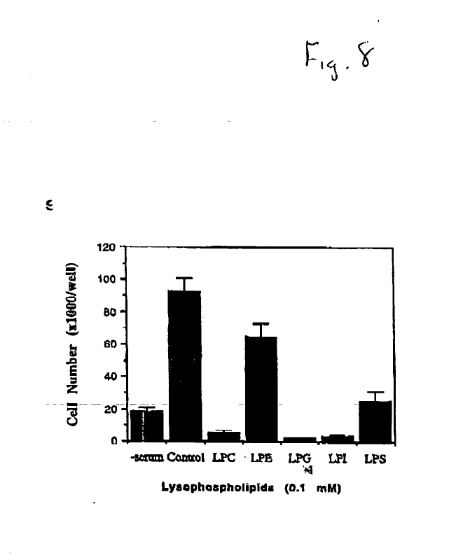

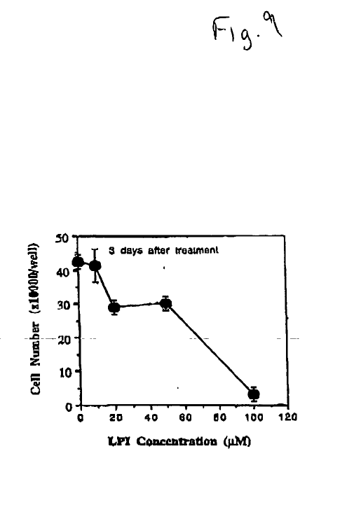

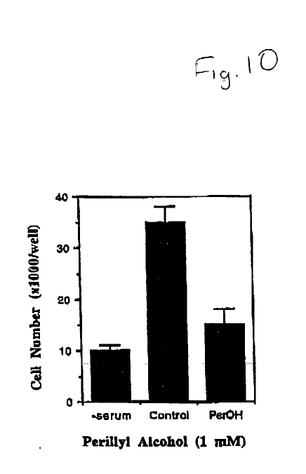

The influence of lysophospholipids on the inhibition of cancer cell growth was investigated. For this purpose, human prostate cancer cell line PC-3 was used. PC-3 cells were cultured using standard RPMI 1640 medium supplemented with 5% fetal bovine serum (FBS). The composition of RPMI 1640 included inorganic salts, glucose, amino acids and vitamins (see Rajesh, D., et al., Molecular Pharmacology 56:515-525 (1999) for details). PC-3 cells were cultured in the presence of various presence of various phospholipids. At appropriate times after treatment the cells were treated by trypsin EDTA treatment, washed twice in an ice cold buffer and analyzed for cell viability after staining with trypan blue (Sawicki, W., et al., Stain Technology 42:143 (1967)). FIG. 7 shows that only the lyso form of the phospholipid (LPI) was able to inhibit the PC-3 cell growth. FIG. 8 shows that several lysophospholipids (LPC, LPG, LPI) are able to retard the growth of PC-3 cells. FIG. 9 shows that the inhibition of PC-3 cell growth by LPI is concentration dependent and that 100 μM concentration is very inhibitory for PC-3 cell growth. FIG. 10 shows that perillyl alcohol is not as strong inhibitor of PC-3 cell growth as compared to LPI even at the 0 times high concentration (1 mM) than LPI.

All references cited herein are hereby incorporated by reference.

The present invention is illustrated by way of the foregoing description and examples. The foregoing description is intended as a non-limiting illustration, since many variations will become apparent to those skilled in the art in view thereof. It is intended that all such variations within the scope and spirit of the appended claims be embraced thereby.

Changes can be made to the composition, operation and arrangement of the method of the present invention described herein without departing from the concept and scope of the invention as defined in the following claims.

What is claimed is:

1. A method for inhibiting phospholipase D activity in a mammal, the method comprising the step of: administering to a mammal an effective amount of a composition to inhibit phospholipase D activity, the composition comprising at least one lysophospholipid selected from the group consisting of:
lysophosphatidylglycerol, lysophosphatidylinositol and lysophosphatidylserine.

2. The method of claim 1 wherein the composition is administered intravenously.

3. The method of claim 1 wherein the composition is administered subcutaneously.

4. The method of claim 1 wherein the composition is administered orally.

5. The method of claim 1 wherein the composition is applied topically.

6. A method for inhibiting the growth of tumor cells in a mammal, the method comprising the step of: administering to a mammal an effective amount of a composition to inhibit the growth of the tumor cells, wherein the composition comprises at least one lysophospholipid selected from the group consisting of:
lysophosphatidylglycerol, lysophosphatidylinositol and lysophosphatidylserine.

7. The method of claim 6 wherein the tumor cells are prostate carcinoma tumor cells, melanoma tumor cells, glial brain tumor cells, Kaposi's sarcoma tumor or lymphoma tumor cells, lung adenocarcinoma tumor cells, breast cancer tumor cells, osteosarcoma tumor cells, fibrosarcoma tumor cells, or squamous cancer tumor cells.

8. The method of claim 6 wherein the composition is administered intravenously.

9. The method of claim 6 wherein the composition is administered subcutaneously.

10. The method of claim 6 wherein the composition is administered orally.

11. The method of claim 6 wherein the composition is applied topically.
